(19) United States
(12) Patent Application Publication
(10) Pub. No.: US 2026/0211016 A1
(43) Pub. Date: Jul. 23, 2026

(54) Estimation Apparatus and Estimation Method

(71) Applicants: SHIMADZU CORPORATION, Kyoto-shi (JP); The University of Osaka, Suita-shi (JP)

(72) Inventors: Taichi TOMONO, Kyoto-shi (JP); Takashi WASHIO, Suita-shi (JP); Satoshi SAITO, Suita-shi (JP)

(21) Appl. No.: 19/418,529

(22) Filed: Dec. 12, 2025

(30) Foreign Application Priority Data
Jan. 20, 2025 (JP) 2025-007822

(51) Int. Cl. G01R 23/16; G01N 21/31

(52) U.S. Cl. CPC G01R 23/16; G01N 21/314

(57) ABSTRACT

An estimation apparatus: generates a measured spectrum indicating a signal intensity for a spectrum parameter value based on analytical data of a component; generates, based on an estimated physical parameter value, an estimated spectrum indicating a signal intensity for the spectrum parameter value; performs convolutional processing using a point spread function, which increases a width of at least one spectrum of the measured spectrum and the estimated spectrum to a first width, and compares the measured spectrum with the estimated spectrum; and estimates a physical parameter value of the component based on a comparison result between the measured spectrum and the estimated spectrum.

### CROSS REFERENCE TO RELATED APPLICATIONS

This nonprovisional application is based on Japanese Patent Application No. 2025-007822 filed on Jan. 20, 2025 with the Japan Patent Office, the entire contents of which are hereby incorporated by reference.

### BACKGROUND OF THE INVENTION

### Field of the Invention

The present disclosure relates to an estimation apparatus and an estimation method for estimating a physical parameter value of a component contained in a sample.

### Description of the Background Art

Conventionally, techniques for analyzing components of compounds contained in a sample have been known. For example, various analytical methods have been known, such as mass spectrometry, nuclear magnetic resonance (NMR), Raman spectroscopy, infrared spectroscopy, X-ray photoelectron spectroscopy (XPS), X-ray diffraction (XRD), X-ray fluorescence (XRF), energy dispersive X-ray spectroscopy (EDX), or X-ray absorption spectroscopy (XAS).

For example, in mass spectrometry, compounds contained in a sample are ionized, the ionized components are separated according to a mass-to-charge ratio, and a spectrum indicating the signal intensity for the separated component is obtained. An analyst can identify the components of the sample by analyzing the spectrum. For example, in manufacture of antibody drugs or nucleic acid drugs, different impurities are generated, and these impurities can lead to a reduction in drug stability, pharmacokinetics, or drug efficacy. Thus, distinguishing among these impurities contained in the drug through the above-described spectral analysis and taking countermeasures is beneficial to development and quality assurance of drugs. However, accurately distinguishing components based on spectra is generally difficult due to the complexity of the spectra.

Therefore, in recent years, measurement informatics has emerged that applies a physical model based on Bayesian estimation or deep learning to estimate a physical parameter value of a component hidden behind a spectrum. For example, an algorithm for estimating a physical parameter value of a component includes processing of inputting a variable of a physical parameter value, such as the number of components, a monoisotopic mass, or the number of functional groups (ions), into a predetermined calculation formula, and generating an estimated spectrum based on the obtained calculation result. The resultant estimated spectrum is compared with a measured spectrum obtained by actually measuring the component, and if the spectra do not match each other, the variable is modified, and an estimated spectrum is generated again using the modified variable. By repeating comparison and modification of the variable as described above, a physical model can be built that is capable of accurately estimating an estimated spectrum corresponding to the physical parameter value of the component contained in the sample.

However, even if a posterior probability or a loss function is calculated by comparing the estimated spectrum estimated from the physical model with the measured spectrum obtained from the actual measurement using a squared error, an L1 norm, a cosine similarity, or the like while changing the physical parameter value, the likelihood thereof will not change. In other words, the gradient of a likelihood function may vanish, making it impossible to learn the physical model. For example, a spectrograph is assumed here that shows a plurality of spectra in a graph with a mass-to-charge ratio or a wavelength on the horizontal axis and a signal intensity on the vertical axis. In such a spectrograph, steep spectra with a signal intensity corresponding to the mass-to-charge ratio or wavelength at which components were detected are sparsely positioned in the horizontal axis direction. Consequently, merely shifting the estimated spectrum slightly in the horizontal axis direction by modifying the physical parameter value does not overlay the estimated spectrum on the measured spectrum, and the gradient is not formed for the likelihood of the posterior probability or the loss function. This makes it difficult to build a physical model that accurately estimates a physical parameter value of a component contained in a to-be-analyzed sample.

Herein, as disclosed in “Bayesian Spectroscopy with a Replica Exchange Monte Carlo Method on an Excitonic Absorption Spectrum of a Cu₂O Thin Crystal”, “Replica-Exchange Monte Carlo Method Incorporating Auto-tuning Algorithm Based on Acceptance Ratios for Effective Bayesian Spectroscopy”, and “Bayesian Deconvolution of Mass and Ion Mobility Spectra: From Binary Interactions to Polydisperse Ensembles”, Bayesian spectroscopy using the replica-exchange Monte Carlo method is proposed as a way to prevent falling into a local solution or a vanishing gradient.

### SUMMARY OF THE INVENTION

However, the use of the above-described technique leads to an extremely high computational cost, and thus, is impractical for an analysis involving large amounts of data.

The present disclosure has been made to solve the above problem. An object of the present disclosure is to provide a technique of accurately estimating a physical parameter value of a component contained in a to-be-analyzed sample.

An estimation apparatus according to an aspect of the present disclosure includes: a data acquisition unit that acquires analytical data of a component; a computing unit that estimates a physical parameter value of the component based on the analytical data acquired by the data acquisition unit; and a display unit that displays the physical parameter value of the component estimated by the computing unit. The computing unit: generates a measured spectrum indicating a signal intensity for a spectrum parameter value based on the analytical data; generates an estimated spectrum indicating a signal intensity for the spectrum parameter value based on an estimated physical parameter value; performs convolutional processing using a point spread function, which increases a width of at least one spectrum of the measured spectrum and the estimated spectrum to a first width, and compares the measured spectrum with the estimated spectrum; and estimates the physical parameter value of the component based on a comparison result between the measured spectrum and the estimated spectrum.

An estimation method according to another aspect of the present disclosure includes: generating, based on analytical data of a component, a measured spectrum indicating a signal intensity for a spectrum parameter value; generating, based on an estimated physical parameter value, an estimated spectrum indicating a signal intensity for the spectrum parameter value; performing convolutional processing using a point spread function, which increases a width of at least one spectrum of the measured spectrum and the estimated spectrum to a first width, and comparing the measured spectrum with the estimated spectrum; and estimating a physical parameter value of the component based on a comparison result between the measured spectrum and the estimated spectrum.

An estimation apparatus according to still another aspect of the present disclosure includes: a data acquisition unit that acquires analytical data of a component; a computing unit that estimates a physical parameter value of the component based on the analytical data acquired by the data acquisition unit; and a display unit that displays the physical parameter value of the component estimated by the computing unit. In estimation of the physical parameter value of the component, the computing unit generates, based on the analytical data, a measured spectrum indicating a signal intensity for a spectrum parameter value and estimates the physical parameter value of the component based on the measured spectrum using an estimation model. In training of the estimation model, the computing unit: determines a first estimated physical parameter value; generates, based on the first estimated physical parameter value, a first estimated spectrum indicating a signal intensity for the spectrum parameter value; estimates a second estimated physical parameter value based on the first estimated spectrum using the estimation model; generates, based on the second estimated physical parameter value, a second estimated spectrum indicating a signal intensity for the spectrum parameter value; performs convolutional processing using a point spread function, which increases a width of at least one spectrum of the first estimated spectrum and the second estimated spectrum, and compares the first estimated spectrum with the second estimated spectrum; and trains the estimation model based on a comparison result between the first estimated spectrum and the second estimated spectrum.

An estimation method according to still another aspect of the present disclosure includes, in estimation of a physical parameter value of a component: generating, based on analytical data of a component, a measured spectrum indicating a signal intensity for a spectrum parameter value; and estimating, based on the measured spectrum, a physical parameter value of the component using an estimated model. The estimation method includes, in training of the estimation model: determining a first estimated physical parameter value; generating, based on the first estimated physical parameter value, a first estimated spectrum indicating a signal intensity for the spectrum parameter value; estimating a second estimated physical parameter value based on the first estimated spectrum using the estimation model; generating, based on the second estimated physical parameter value, a second estimated spectrum indicating a signal intensity for the spectrum parameter value; performing convolutional processing using a point spread function, which increases a width of at least one spectrum of the first estimated spectrum and the second estimated spectrum, and comparing the first estimated spectrum with the second estimated spectrum; and training the estimation model based on a comparison result between the first estimated spectrum and the second estimated spectrum.

The foregoing and other objects, features, aspects, and advantages of the present invention will become more apparent from the following detailed description of the present invention when taken in conjunction with the accompanying drawings.

### BRIEF DESCRIPTION OF THE DRAWINGS

FIG. 1 is a diagram showing a configuration of an analysis system according to Embodiment 1.

FIG. 2 is a diagram illustrating an overview of estimation processing performed by an estimation apparatus according to Embodiment 1.

FIG. 3 is a diagram showing an example comparison between a measured spectrum and an estimated spectrum.

FIG. 4 is a flowchart of a main process in the estimation processing performed by the estimation apparatus according to Embodiment 1.

FIG. 5 is a flowchart of a subprocess in the estimation processing performed by the estimation apparatus according to Embodiment 1.

FIG. 6 is a diagram illustrating an overview of estimation processing performed by an estimation apparatus according to Embodiment 2.

FIG. 7 is a diagram illustrating an overview of training processing for an estimation model performed by an estimation apparatus according to Embodiment 3.

FIG. 8 is a flowchart of a main process in the training processing performed by the estimation apparatus according to Embodiment 3.

FIG. 9 is a flowchart of a subprocess in the training processing performed by the estimation apparatus according to Embodiment 3.

FIG. 10 is a diagram illustrating an overview of estimation processing performed by the estimation apparatus according to Embodiment 3.

FIG. 11 is a flowchart showing the estimation processing performed by the estimation apparatus according to Embodiment 3.

### DESCRIPTION OF THE PREFERRED EMBODIMENTS

### Embodiment 1

Embodiment 1 will be described in detail with reference to the drawings. The same or corresponding parts in the drawings are denoted by the same reference signs, and description thereof will not be repeated in principle.

### [Configuration of Analysis System]

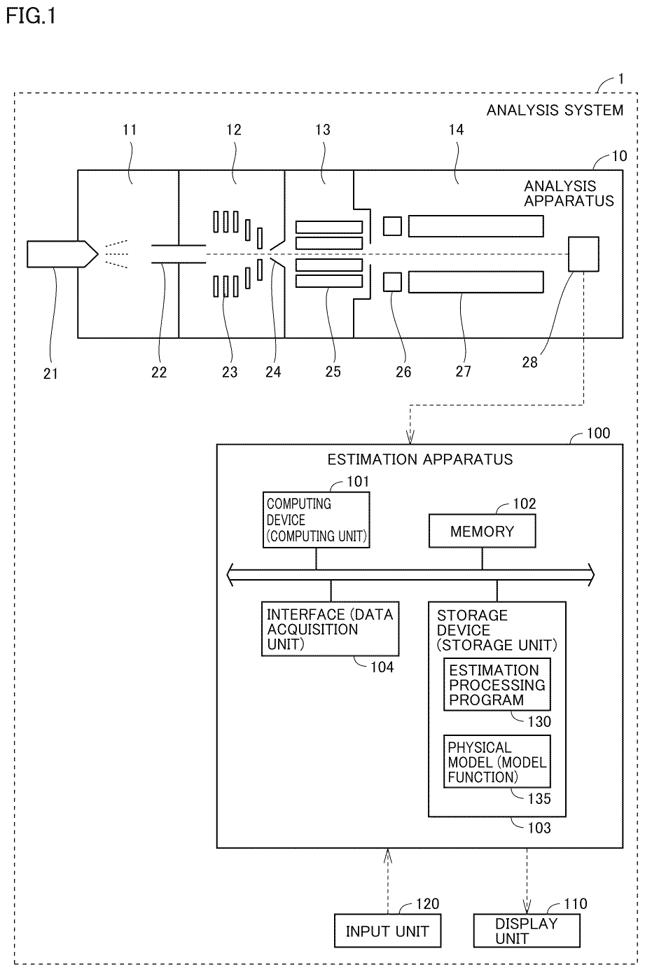

FIG. 1 is a diagram showing a configuration of an analysis system 1 according to Embodiment 1. As shown in FIG. 1, analysis system 1 includes an analysis apparatus 10 and an estimation apparatus 100.

In Embodiment 1, analysis apparatus 10 is a mass spectrometry apparatus. Analysis apparatus 10 includes an ionization chamber 11, a first intermediate chamber 12, a second intermediate chamber 13, and an analysis chamber 14.

Ionization chamber 11 includes a probe 21 and a capillary 22. The interior of ionization chamber 11 has an atmospheric pressure. Ionization chamber 11 communicates with first intermediate chamber 12 of a subsequent stage through capillary 22 having a small diameter. Probe 21 introduces a sample into analysis apparatus 10. A minute electrically-charged droplet of the sample is refined while being fragmented by the action of an electrostatic force, and lipids of the sample are ionized during evaporation of a solvent. The generated ions are introduced into first intermediate chamber 12 through capillary 22.

First intermediate chamber 12 includes a first ion guide 23 and a skimmer 24. The interior of first intermediate chamber 12 is a high vacuum. First intermediate chamber 12 communicates with second intermediate chamber 13 of a subsequent stage through a small hole bored in the top of skimmer 24. First ion guide 23 transports the ions introduced from ionization chamber 11 of a preceding stage through skimmer 24 while converging the ions.

Second intermediate chamber 13 includes a second ion guide 25. The interior of second intermediate chamber 13 is a high vacuum. Second ion guide 25 transports ions introduced from first intermediate chamber 12 of a preceding stage while converging the ions.

Analysis chamber 14 includes a pre-rod electrode 26, a main rod electrode 27, and an ion detector 28. The interior of analysis chamber 14 has an atmospheric pressure. Analysis chamber 14 separates ions by mass and detects each separated ion.

Pre-rod electrode 26 corrects electric field distortions at the inlet end and assists the action of main rod electrode 27. Main rod electrode 27 separates ions according to a mass-to-charge ratio.

Ion detector 28 is, for example, a detector of pulse counting type and generates pulse signals corresponding to the number of incident ions as analytical data. This analytical data is output to estimation apparatus 100.

Analysis apparatus 10, which is the mass spectrometry apparatus as described above, is applicable to a liquid chromatography mass spectrometer (LC-MS). Analysis apparatus 10 is not limited to a mass spectrometry apparatus and may be an apparatus that analyzes components of a sample by any other analytical method such as magnetic resonance, Raman spectroscopy, infrared spectroscopy, X-ray photoelectron spectroscopy, X-ray diffraction, X-ray fluorescence analysis, energy-dispersive X-ray spectroscopy, or X-ray absorption spectroscopy. Analysis apparatus 10 may be any analysis apparatus for obtaining a spectrograph in which a plurality of steep spectra are sparsely positioned.

Estimation apparatus 100 may be a general-purpose computer or a computer dedicated to analysis system 1 for processing analytical data from analysis apparatus 10. Estimation apparatus 100 includes a computing device 101, a memory 102, a storage device 103, and an interface 104.

Computing device 101 is an example of the “computing unit”. Computing device 101 is a computing entity (computer) that performs various types of processing by executing various programs. Computing device 101 includes, for example, a processor such as a central processing unit (CPU), a micro processing unit (MPU), a tensor processing unit (TPU), or a graphics processing unit (GPU). The processor, which is an example of computing device 101, has the function of performing various types of processing by executing the programs, but some or all of these functions may be implemented using a dedicated hardware circuit such as an application specific integrated circuit (ASIC) or a field programmable gate array (FPGA). The “processor” is not limited to a narrowly-defined processor that performs processing in a stored program manner, such as a CPU, an MPU, a TPU, or a GPU, and may also include a hardwired circuit such as an ASIC or an FPGA. Thus, the “processor”, which is an example of computing device 101, may also be read as computing processing circuitry in which processing is defined in advance by a computer-readable code and/or hardwired circuitry. Computing device 101 may be composed of one chip or a plurality of chips. Further, the processor and associated processing circuitry may be composed of a plurality of computers interconnected in a wired or wireless manner, through a local area network, a wireless network, or the like. The processor and associated processing circuitry may also be configured of a cloud computer that performs computations remotely based on input data and outputs a computing result to any other remotely-positioned device.

Memory 102 includes a volatile storage area (e.g., working area) for temporarily storing a program code, a working memory, or the like when computing device 101 executes various programs. Examples of memory 102 include volatile memories such as a dynamic random access memory (DRAM) and a static random access memory (SRAM), or non-volatile memories such as a read only memory (ROM) and a flash memory. Memory 102 may also be read as memory processing circuitry. Storage device 103 is an example of the “storage unit”. Storage device 103 stores various programs, various data, or the like executed by computing device 101.

Storage device 103 may be one or more non-transitory computer-readable media or one or more computer-readable storage media. Examples of storage device 103 include a hard disk drive (HDD) and a solid state drive (SSD). Storage device 103 according to the embodiment stores an estimation processing program 130 for performing estimation processing of estimating a physical parameter value of a component based on analytical data acquired from analysis apparatus 10 by computing device 101. Further, storage device 103 stores a physical model 135 composed of model functions used in the estimation processing. Storage device 103 may also be read as storage processing circuitry.

Interface 104 is an example of the “data acquisition unit”. Interface 104 transmits and receives data to and from an external device or external equipment via wired communication or wireless communication. For example, interface 104 acquires analytical data output from analysis apparatus 10 by communicating with analysis apparatus 10. Further, interface 104 may be a communication device that communicates with a cloud server (not shown) to transmit analytical data acquired from analysis apparatus 10 to the cloud server, or to transmit the results of estimation processing performed by computing device 101 to the cloud server. Additionally, interface 104 may transmit and receive data to and from display unit 110 or input unit 120, which are user interfaces, via wired communication or wireless communication. Estimation apparatus 100 is not limited to a single interface 104 and may include a plurality of interfaces 104 depending on the number of communication targets. Interface 104 may also be read as input processing circuitry or output processing circuitry.

Display unit 110, which is, for example, a display configured of an LCD panel, displays a physical parameter value of a component estimated by estimation apparatus 100. Input unit 120, which is, for example, a pointing device such as a keyboard or a mouse, accepts a command from the user. When a touch panel is used as the user interface, display unit 110 and input unit 120 may be integrally formed. Display unit 110 and input unit 120 may be components included in estimation apparatus 100.

In analysis system 1 configured as described above, estimation apparatus 100 acquires analytical data of a component contained in a sample from analysis apparatus 10 via interface 104. Based on the acquired analytical data, estimation apparatus 100 generates a spectrum indicating a signal intensity for a spectral parameter value.

### Example of Spectrograph

A spectrograph generated by analysis system 1 will be described. Estimation apparatus 100 can generate a spectrograph including at least one spectrum based on the analytical data acquired from analysis apparatus 10. For example, when analysis apparatus 10 is configured to detect the ion intensity of components (various compounds) contained in a sample as a signal intensity for each mass-to-charge ratio, estimation apparatus 100 can generate a spectrograph indicating a signal intensity for the mass-to-charge ratio based on the analytical data acquired from analysis apparatus 10. Specifically, in a graph with a mass-to-charge ratio on the horizontal axis and a signal intensity on the vertical axis, a plurality of steep spectra having signal intensity for each specific component, are sparsely positioned and displayed. Alternatively, when analysis apparatus 10 is configured to detect the absorbance of various compounds contained in the sample as a signal intensity for each wavelength, estimation apparatus 100 can generate a spectrograph indicating a signal intensity for a wavelength based on the analytical data acquired from analysis apparatus 10. Specifically, in a graph with a wavelength on the horizontal axis and a signal intensity on the vertical axis, a plurality of steep spectra with a signal intensity for each component are sparsely positioned and displayed. The value (mass-to-charge ratio, wavelength) shown on the horizontal axis as described above is also referred to as a “spectral parameter value”.

### [Overview of Estimation Processing]

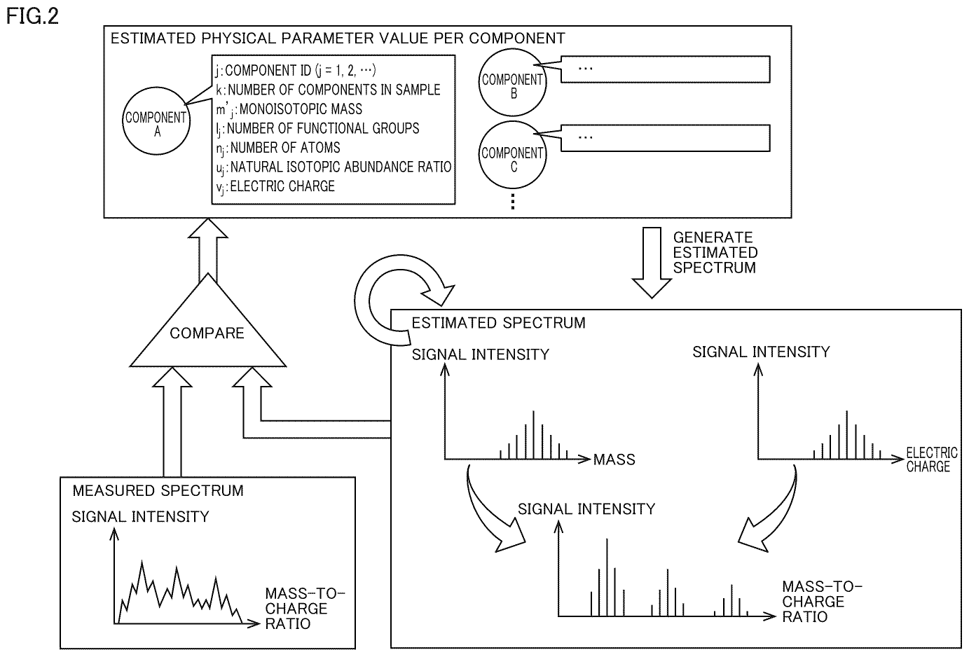

FIG. 2 is a diagram illustrating an overview of estimation processing performed by estimation apparatus 100 according to Embodiment 1. Estimation apparatus 100 is configured to estimate a physical parameter value of a component hidden behind at least one spectrum included in the spectrograph using physical model 135 employing Bayesian estimation.

Specifically, as shown in FIG. 2, it is assumed that each of the plurality of components (in this example, components A, B, C . . . ) has a unique physical parameter value (physical parameter set). The physical parameter value (also corresponding to an estimated physical parameter value) of each component includes a component ID for identifying a component, the number of components contained in the sample, a monoisotopic mass, the number of functional groups, a charging rate of a functional group, the number of atoms, a natural isotopic abundance ratio, and an electric charge. The physical parameter value may include at least one of a component ID, the number of components, a monoisotopic mass, the number of functional groups, a charging rate of a functional group, the number of atoms, a natural isotopic abundance ratio, and an electric charge. The physical parameter value of each component may be stored in memory device 103 of estimation apparatus 100 or obtained externally via interface 104.

First, the analyst actually measures a to-be-analyzed sample using analysis apparatus 10. Analytical data from analysis apparatus 10 is input to estimation apparatus 100. Estimation apparatus 100 generates a measured spectrum indicating a signal intensity for a spectral parameter value based on the analytical data acquired from analysis apparatus 10. In the example of FIG. 2, estimation apparatus 100 generates, as a measured spectrum, a mass spectrum indicating a signal intensity for a mass or a mass-to-charge ratio of an ionized component.

Next, estimation apparatus 100 determines a to-be-compared number of components. For example, estimation apparatus 100 determines “1” as the number of components. Estimation apparatus 100 determines a to-be-compared physical parameter value for the number of components “1”. The to-be-compared physical parameter value is an example of the “estimated physical parameter value”. At this time, estimation apparatus 100 selects several estimated physical parameter values (physical parameter sets) from a plurality of estimated physical parameter values corresponding to one component, and uses the concept of prior distribution in the selection. For example, the number of components for each of the plurality of estimated physical parameter values is determined in advance by the prior distribution. Data on this prior distribution may be stored in storage device 103 of estimation apparatus 100 or acquired externally via interface 104. For example, estimation apparatus 100 preferentially selects an estimated physical parameter value with a higher number of components using a graph of the prior distribution with each estimated physical parameter value on the horizontal axis and the number of components on the vertical axis.

Estimation apparatus 100 generates an estimated spectrum using physical model 135 described below, based on the determined estimated physical parameter value (physical parameter set). For example, estimation apparatus 100 generates an estimated spectrum showing a signal intensity corresponding to the mass of the component using Equation (1) below.

$$[ Math 1 ]$$

$${\tilde{p}}_{j} ( {ω}_{j} ) = ( \begin{matrix}{n}_{j} \\ {ω}_{j}\end{matrix} ) {{{u}_{j}}^{{ω}_{j}} ( 1 - {u}_{j} )}^{{n}_{j} - {ω}_{j}} \qquad ( 1 )$$

In Equation (1), ωⱼ is expressed by Equation (2) below.

$$[ Math 2 ]$$

$${ω}_{j} = round ( \frac{m - {m}_{j}^{′}}{ε} ) \qquad ( 2 )$$

In Equation (2), m represents a variable in a mass space, m′ⱼ represents a monoisotopic mass, and ¿ represents a mass of a neutron (1.008664 Da). Also, in Equation (1), nⱼ represents the number of atoms, and uⱼ represents a natural isotopic abundance ratio.

Estimation apparatus 100 generates a spectrum indicating a signal intensity corresponding to an electric charge of a component using Equation (3) below, based on the acquired estimated physical parameter value.

$$[ Math 3 ]$$

$${\tilde{q}}_{j} ( z ) = ( \begin{matrix}{l}_{j} \\ z\end{matrix} ) {{{v}_{j}}^{z} ( 1 - {v}_{j} )}^{{l}_{j} - z} \qquad ( 3 )$$

In Equation (3), vⱼ represents an electric charge, Z is a variable representing an absolute value of the electric charge, which is an integer, and lⱼ represents the number of functional groups. In Equations (1) to (3), the subscript j represents a component ID.

Usually, the spectrum obtained from analysis apparatus 10, which is a mass spectrometry apparatus, shows a mass-to-charge ratio (m/z) along the horizontal axis. Herein, when φ is defined as a variable representing m/z, the total number of ions belonging to component j is represented by lⱼ. Each ion is indexed by ij. The mass and electric charge of each ion ij are expressed by Equations (4) and (5), respectively.

$$[ Math 4 ]$$

$${ω}_{{i}_{j}} \sim {\tilde{p}}_{j} \qquad ( 4 )$$

$$[ Math 5 ]$$

$${z}_{{i}_{j}} \sim {\tilde{q}}_{j} \qquad ( 5 )$$

When ion ij is detected, an observed ideal spectrum is expressed by Equation (6) below. In Equation (6), δ is a Kronecker delta function.

$$[ Math 6 ]$$

$$δ ( φ - ( {m}_{j}^{′} + ε {ω}_{{i}_{j}} ) / {z}_{{i}_{j}} ) \qquad ( 6 )$$

Regardless of the charge state or mass, a single ion contributes to the observed spectrum as a single delta function. Therefore, the ideal spectrum formed of a set of ions (ij=1 to Ij) is expressed by Equation (7) below.

$$[ Math 7 ]$$

$${D}_{j} ( φ ) = {\sum }_{{i}_{j} = 1}^{{l}_{j}} δ ( φ - ( {m}_{j}^{′} + {εω}_{{i}_{j}} ) / {z}_{{i}_{j}} ) \qquad ( 7 )$$

The theoretical probability distribution of ions belonging to a structural component j on the φ-axis (the horizontal axis corresponding to m/z) is expressed by Equation (8) below and is determined solely by ωj and z that are independent of each other. This independence arises from the fact that ωj is a function of m and a chemical property z is hardly affected by an isotopic mass m. Therefore, the theoretical probability distribution of Equation (8) is obtained by summation of the product of the probability of ωj, the probability of z, and the Kronecker delta function of Equation (6) over ωj and z.

$$[ Math 8 ]$$

$${U}_{j} ( φ ) = {\sum }_{z = 1}^{∞} {\sum }_{{ω}_{i} = 1}^{∞} {\tilde{p}}_{j} ( {ω}_{j} ) \cdot {q}_{j} ( z ) \cdot δ ( φ - ( {m}_{j}^{′} + {εω}_{j} ) / z ) \qquad ( 8 )$$

Regardless of the state of charge or the mass, a single ion contributes as a single delta function. Therefore, the observed ion spectrum is proportional to the probability distribution of ions along the φ axis. According to the Glivenko-Cantelli theorem, as the sample size increases, the empirical spectrum expressed by Equation (9) below converges uniformly to the theoretical probability distribution of Equation (8). Thus, the ideal empirical spectrum of component j is approximated by the theoretical probability distribution as expressed by Equation (9).

$$[ Math 9 ]$$

$${D}_{j} ( φ ) = {\sum }_{{i}_{j} = 1}^{{l}_{j}} δ ( φ - ( {m}_{j}^{′} + {εω}_{{i}_{j}} ) / {z}_{{i}_{j}} ) \sim {l}_{j} \cdot {U}_{j} ( φ ) ( {I}_{j} ≫ 1 ) \qquad ( 9 )$$

Due to the point spread of a response R(φ) of analysis apparatus 10, the observed spectrum becomes the convolution of the approximate spectrum of component j, represented by IjφUj(φ), and R(φ), resulting in Ij·(Uj*R)(φ). Thus, summation of the spectra of all the components contained in the sample yields an estimated spectrum as expressed by Equation (10) below. Equation (10) corresponds to the model function of physical model 135 for generating an estimated spectrum based on the estimated physical parameter value.

$$[ Math 10 ]$$

$${\hat{S}}_{ms} ( φ ) = {\sum }_{j = 1}^{k} {I}_{j} \cdot ( {U}_{j} * R ) ( φ ) \qquad ( 10 )$$

Estimation apparatus 100 compares the estimated spectrum expressed by Equation (10) with the measured spectrum and estimates a physical parameter value of a component corresponding to the measured spectrum using Bayesian estimation (Stochastic Variational Inference: SVI). Specifically, estimation apparatus 100 calculates a posterior probability between an estimated spectrum and a measured spectrum using, for example, a squared error, an L1 norm, or a cosine similarity, and then, regards the number of components and the estimated physical parameter value corresponding to the estimated spectrum at which the posterior probability is maximized, as the number of components and the physical parameter value of the to-be-analyzed sample.

For example, estimation apparatus 100 uses Bayesian estimation to infer a maximum posterior probability for each estimated physical parameter value and determines the number of components and an estimated physical parameter value corresponding to the estimated spectrum at which the posterior probability is maximized. In this optimization problem, the gradient descent method, widely used in machine learning, can be used to find an estimated physical parameter value that maximizes a likelihood function. For example, estimation apparatus 100 uses stochastic gradient descent (SGD) or adaptive moment estimation (Adam), which are types of gradient descent methods, to find an estimated physical parameter value that maximizes a likelihood function. For example, when the steepest descent method is used, estimation apparatus 100 differentiates the posterior probability between a measured spectrum and an estimated spectrum with respect to an internal parameter and updates the internal parameter based on a calculated value. Repeatedly updating the internal parameter in this manner enables determination of the number of components and an estimated physical parameter value corresponding to the estimated spectrum at which the posterior probability is maximized.

Herein, the spectrum indicating the signal intensity corresponding to the mass-to-charge ratio is steep. Further, a plurality of such steep spectra are sparsely positioned in the horizontal axis direction representing the mass-to-charge ratio. Consequently, even if a posterior probability is calculated by comparing the estimated spectrum with the measured spectrum while changing the estimated physical parameter value, the gradient of the likelihood function vanishes. Specifically, the estimated spectrum generated based on the estimated physical parameter value should approach the measured spectrum generated based on the actual physical parameter value, and the posterior probability calculated based on the estimated spectrum and the measured spectrum should increase. Further, as the estimated physical parameter value approaches the actual physical parameter value, the gradient of the likelihood function should arise. In a collection of spectra like mass spectra, however, a plurality of steep spectra are sparsely positioned in the horizontal axis direction. Consequently, even if the estimated physical parameter value approaches the actual physical parameter value, both the spectra may not overlap each other. In this case, the gradient of the likelihood function vanishes. If the estimated spectra and the measured spectra in this state are compared with each other without any change, the gradient of the likelihood function vanishes, making it difficult to build physical model 135 that appropriately estimates a physical parameter value.

Thus, in order to create a suitable gradient in the likelihood function, estimation apparatus 100 performs convolution processing using a point spread function (PSF) on at least one spectrum of the measured spectrum and the estimated spectrum, thereby transforming the shape of the at least one spectrum into a Gaussian shape. For example, estimation apparatus 100 may perform convolution processing using a point spread function on both the measured spectrum and the estimated spectrum to transform the shape of each of the measured spectrum and the estimated spectrum into a Gaussian shape, and then, compares these spectra. Estimation apparatus 100 may perform convolution processing using the point spread function only on the measured spectrum, or perform convolution processing using the point spread function only on the estimated spectrum.

The point spread function is expressed by Equation (11) below.

$$[ Math 11 ]$$

$$g ( φ ) = \frac{1}{\sqrt{2 π {T}^{2}}} exp ( - \frac{1}{2 {T}^{2}} {( φ )}^{2} ) \qquad ( 11 )$$

In Equation (12), T represents a variance of the Gaussian distribution and is expressed by Equation (12) below.

$$[ Math 12 ]$$

$$T = λ {( \frac{{s}_{max} - s}{{s}_{max}} )}^{4} ( s = 0 , 1 , 2 , … , {s}_{max} ) \qquad ( 12 )$$

In Equation (12), 2 represents a coefficient and is set in advance. When S=Sₘₐₓ, the spectrum before convolution matches the pre-convolution spectrum after convolution. Gradually decreasing S away from Sₘₐₓ causes the Gaussian shape of the spectrum to spread.

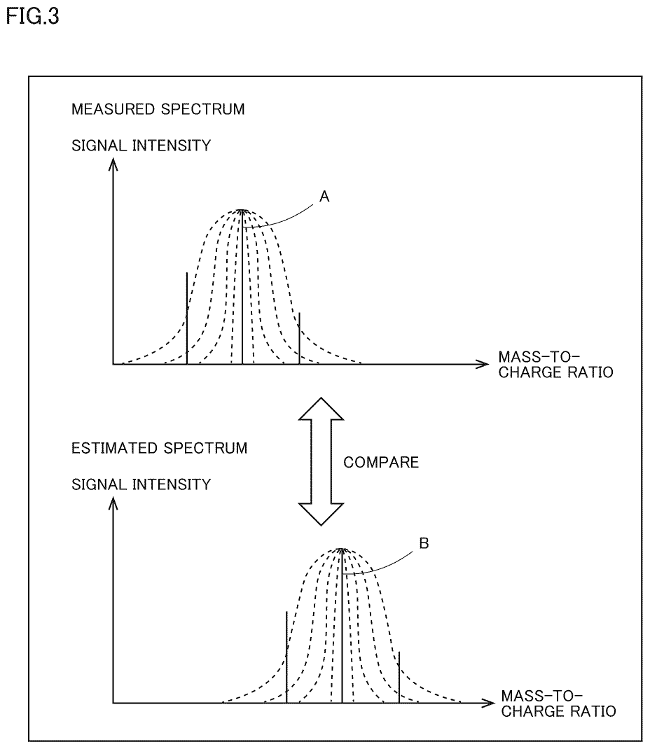

For example, FIG. 3 is a diagram showing an example comparison between a measured spectrum and an estimated spectrum. As shown in FIG. 3, when a measured spectrum A is compared with an estimated spectrum B, before convolution processing is performed, the spectra do not overlap each other due to a shift occurring in the horizontal axis direction. In contrast, when convolution processing is performed to transform the shape of at least one spectrum of the measured spectrum and the estimated spectrum (in this example, both the measured spectrum and the estimated spectrum) into a Gaussian shape, and the width of the spectrum transformed into the Gaussian shape is spread in the horizontal axis direction, at least part of the measured spectrum can be overlaid on the estimated spectrum. This enables estimation apparatus 100 to compare the measured spectrum with the estimated spectrum to calculate a posterior probability.

The measured spectrum and the estimated spectrum transformed into the Gaussian shape to be blurred are expressed by Equation (13) and Equation (14) below, respectively.

$$[ Math 13 ]$$

$${S}_{obs}^{′} ( φ ) = ( {S}_{obs} * g ) ( φ ) \qquad ( 13 )$$

$$[ Math 14 ]$$

$${\hat{S}}_{ms}^{′} ( φ ) = ( {\hat{S}}_{ms} * g ) ( φ ) \qquad ( 14 )$$

Using these blurred measured spectrum and estimated spectrum, a modified log likelihood is derived, and the logarithm of the posterior probability expressed by Equation (15) below is calculated.

$$[ Math 15 ]$$

$$log ( {P}_{k} ( {S}_{abs}^{′} \vert {θ}_{k} ) ) = - \frac{1}{2 {σ}^{2}} \int {\vert {\hat{S}}_{ms}^{′} ( φ ) - {S}_{abs}^{′} ( φ ) \vert}^{2} d φ + N \cdot log ( σ ) + \frac{N}{2} \cdot log ( 2 π ) \qquad ( 15 )$$

In Equation (15), the modified log likelihood (posterior probability) is defined by Equation (16) below.

$$[ Math 16 ]$$

$$\begin{matrix}\begin{matrix}{LP}_{k}^{′} := log ( {P}_{k} ( {S}_{abs}^{′} \vert {θ}_{k} ) ) + log ( {P}_{k} ( {θ}_{k} ) ) \\ = - \frac{1}{2 {σ}^{2}} \int {\vert {\hat{S}}_{ms}^{′} ( φ ) - {S}_{abs}^{′} ( φ ) \vert}^{2} d φ + N \cdot log ( σ ) + \frac{N}{2} \cdot log ( 2 π )\end{matrix} \\ - {w}_{bic} ( k ) - {w}_{ex} ( k , {m}_{1}^{′} … {m}_{k}^{′} ) .\end{matrix} \qquad ( 16 )$$

Returning to FIG. 2, estimation apparatus 100 determines another to-be-compared estimated physical parameter value from a plurality of estimated physical parameter values defined by prior distribution, based on the comparison result (the posterior probability expressed by Equation (16)) between the measured spectrum and the estimated spectrum. For example, based on the comparison result, estimation apparatus 100 uses Adam to determine the next candidate estimated physical parameter value capable of generating an estimated spectrum closer to the measured spectrum. The estimated spectrum based on the estimated physical parameter value determined in this manner becomes closer to the measured spectrum.

In a first search, estimation apparatus 100 searches for an optimal estimated physical parameter value for generating an estimated spectrum that approaches the measured spectrum while changing the estimated physical parameter value as described above, with the Gaussian shape set to be relatively broad (e.g., with the spectrum width set to a first width). In other words, in the first search, estimation apparatus 100 performs convolution processing using a point spread function, which spreads the width of the estimated spectrum generated based on the estimated physical parameter value to a first width, compares the measured spectrum with the estimated spectrum, and estimates a physical parameter value of a component based on a comparison result. Upon completion of the first search for the estimated physical parameter value, in a second search, estimation apparatus 100 again searches for an optimal estimated physical parameter value for generating an estimated spectrum that approaches the measured spectrum while modifying the estimated physical parameter value, with the Gaussian shape set to be narrower than in the first search (e.g., with the spectrum width set to a second width narrower than the first width). The estimated physical parameter value used in the beginning of the second search is an estimated physical parameter value (any other estimated physical parameter value) finally determined in the first search. In other words, in the second search, estimation apparatus 100 performs convolution processing using a point spread function, which spreads the width of another estimated spectrum generated based on the other estimated physical parameter value to a second width narrower than the first width, compares the measured spectrum with the other estimated spectrum, and estimates a physical parameter value of the component based on a comparison result. In this manner, estimation apparatus 100 searches for an optimal estimated physical parameter value for generating an estimated spectrum that is closest to the measured spectrum while gradually narrowing the Gaussian shape.

Estimation apparatus 100 performs the above-described search for an estimated physical parameter value while varying the number of components. For example, when determining “2” as the number of components, estimation apparatus 100 determines the next to-be-compared estimated physical parameter value for the number of components “2”. Based on the to-be-compared estimated physical parameter value, estimation apparatus 100 generates an estimated spectrum, and compares the estimated spectrum with the measured spectrum while performing convolution processing using a point spread function, thereby calculating a posterior probability. Estimation apparatus 100 searches for an estimated physical parameter value while gradually narrowing the Gaussian shape.

Estimation apparatus 100 performs the above-described estimation processing while gradually increasing the number of components, and calculate a posterior probability each time. Estimation apparatus 100 determines, as the number of components and the physical parameter value of the to-be-analyzed sample, the number of components and the estimated physical parameter value (physical parameter set) used in the calculation of a maximum posterior probability among the plurality of posterior probabilities calculated for each number of components.

### [Flow of Estimation Processing of Estimation Apparatus]

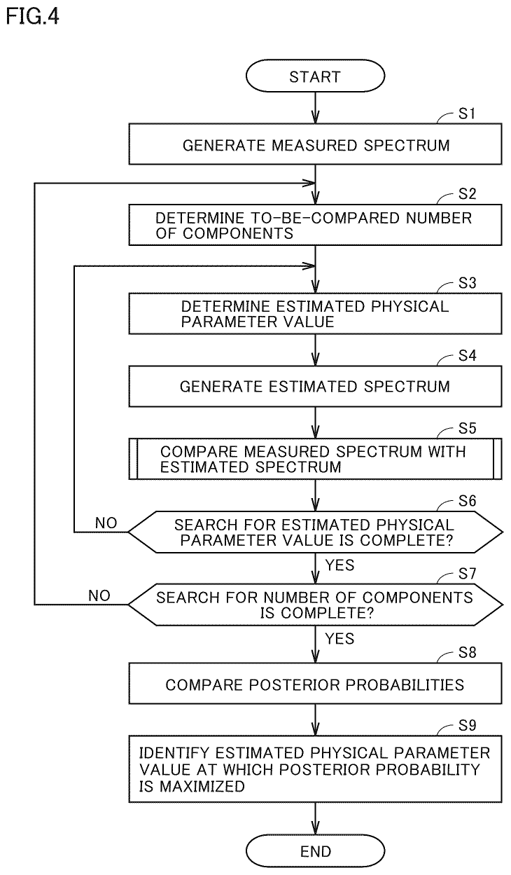

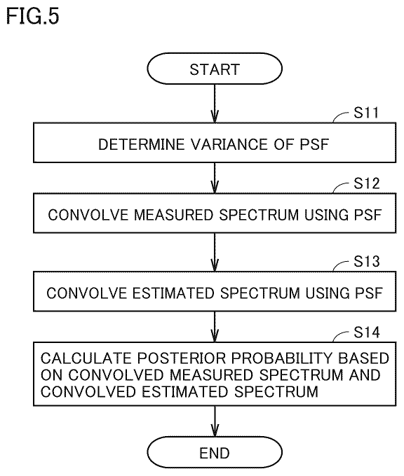

FIG. 4 is a flowchart of a main process in the estimation processing performed by estimation apparatus 100 according to Embodiment 1. FIG. 5 is a flowchart of a subprocess in the estimation processing performed by the estimation apparatus according to Embodiment 1. Each processing step (hereinafter abbreviated as “S”) shown in FIGS. 4 and 5 is realized by computing device 101 executing estimation processing program 130.

As shown in FIG. 4, estimation apparatus 100 generates a measured spectrum indicating a signal intensity for a spectral parameter value based on analytical data acquired from analysis apparatus 10 (S1). Estimation apparatus 100 determines a to-be-compared number of components (S2). For example, in the first estimation, estimation apparatus 100 determines “1” as the number of components.

Estimation apparatus 100 determines a to-be-compared estimated physical parameter value (S3). Estimation apparatus 100 generates an estimated spectrum based on the determined estimated physical parameter value (S4). For example, estimation apparatus 100 uses physical model 135 to generate an estimated spectrum indicating a signal intensity for a mass-to-charge ratio (spectral parameter value) based on a mass and an electric charge, which are the estimated physical parameter values. Estimation apparatus 100 compares the measured spectrum generated in S1 with the estimated spectrum generated in S4 (S5).

The subprocess of the processing in S5 is shown in FIG. 5. Specifically, as shown in FIG. 5, estimation apparatus 100 determines a variance of the point spread function (PSF) (S11). For example, in the first search, estimation apparatus 100 sets the Gaussian shape to be relatively broad.

Estimation apparatus 100 performs convolution processing using a point spread function (PSF) on at least one spectrum of the measured spectrum and the estimated spectrum. Estimation apparatus 100 increases the width of the measured spectrum in the horizontal axis direction by performing convolution processing using a point spread function on the measured spectrum to transform the measured spectrum into a Gaussian shape (S12). Further, estimation apparatus 100 performs convolution processing using a point spread function on the estimated spectrum to transform the estimated spectrum into a Gaussian shape, thereby increasing the width of the estimated spectrum in the horizontal axis direction (S13).

After the convolution processing, estimation apparatus 100 compares the measured spectrum with the estimated spectrum to calculate a posterior probability (S14). For example, estimation apparatus 100 calculates a posterior probability based on the measured spectrum after the convolution processing and the estimated spectrum after the convolution processing.

Returning to FIG. 4, estimation apparatus 100 determines whether the search for an estimated physical parameter value is complete (S6). For example, when having not found an optimal estimated physical parameter value by the comparison between the measured spectrum and the estimated spectrum (NO in S6), estimation apparatus 100 returns to S3. Estimation apparatus 100 then uses, for example, Adam to determine a next to-be-compared estimated physical parameter value (another estimated physical parameter value) based on the posterior probability, and repeats the processing of S4 and subsequent processing.

In contrast, when having found an optimal estimated physical parameter value by the comparison between the measured spectrum and the estimated spectrum (YES in S6), estimation apparatus 100 determines whether the search for the number of components is complete (S7). When the search for the number of components is not complete (NO in S7), estimation apparatus 100 returns to S2, determines a next to-be-compared number of components, and repeats the processing of S3 and subsequent processing. In contrast, when the search for the number of components is complete (YES in S7), estimation apparatus 100 compares the plurality of posterior probabilities calculated for each number of components (S8). As a result of the comparison among the plurality of posterior probabilities, estimation apparatus 100 determines, as the number of components and physical parameter value of the to-be-analyzed sample, the number of components and an estimated physical parameter value (physical parameter set) used in the calculation of the maximum posterior probability (S9). Estimation apparatus 100 then terminates the estimation processing shown in FIG. 4.

As described above, estimation apparatus 100 according to Embodiment 1 can estimate the number of components and the physical parameter value of the to-be-analyzed sample by comparing the estimated spectrum, which is estimated using physical model 135 based on the estimated physical parameter value, with the measured spectrum, which is obtained by measuring the to-be-analyzed sample. Further, when comparing the estimated spectrum with the measured spectrum, estimation apparatus 100 performs convolution processing using a point spread function on at least one spectrum of the measured spectrum and the estimated spectrum and then compares the spectra with each other. This enables estimation apparatus 100 to avoid a situation in which the gradient of the likelihood function vanishes, making it impossible to search for an optimal estimated physical parameter value. Consequently, estimation apparatus 100 can accurately estimate a physical parameter value of a component contained in a to-be-analyzed sample, without increasing a computational cost.

Estimation apparatus 100 determines the estimated physical parameter value using the steepest descent method such as Adam in the example described above, but it may determine the estimated physical parameter value using a method as described below.

For example, estimation apparatus 100 compares a first estimated spectrum generated based on the first estimated physical parameter value with a measured spectrum and calculates a first likelihood. Similarly, estimation apparatus 100 compares a second estimated spectrum generated based on a second estimated physical parameter value with the measured spectrum and calculates a second likelihood. Estimation apparatus 100 compares the first likelihood with the second likelihood to find that the estimated spectrum of the estimated physical parameter value with the higher likelihood is closer to the measured spectrum. Thus, when the first likelihood is higher than the second likelihood, estimation apparatus 100 generates a subsequent third estimated spectrum using the first estimated physical parameter value. By repeating the above-described processing, estimation apparatus 100 considers that the search for the estimated physical parameter value completes as the likelihood between the measured spectrum and the estimated spectrum becomes higher than or equal to a predetermined value, and terminates the processing. Estimation apparatus 100 displays the estimated physical parameter value corresponding to the estimated spectrum at the time of completion of the processing on display unit 110 to present this value to the user.

In the example described above, the measured spectrum and the estimated spectrum are shown in a two-dimensional graph with a mass-to-charge ratio on the horizontal axis and a signal intensity on the vertical axis, but the spectra may also be shown in a three-dimensional or higher-dimensional graph. For example, the measured spectrum and the estimated spectrum may be shown in a three-dimensional graph with a mass-to-charge ratio on the X-axis direction, a wavelength in the Y-axis direction, and a signal intensity in the Z-axis direction. In this case, estimation apparatus 100 may transform the spectrum of a signal intensity for a mass-to-charge ratio, which is represented by the X axis and the Z axis, into a Gaussian shape and transform the spectrum of the signal intensity for the wavelength, which is represented by the Y axis and the Z axis, into a Gaussian shape, and compare the measured spectrum with the estimated spectrum.

### Embodiment 2

Estimation apparatus 100 according to Embodiment 2 will be described in detail with reference to the drawings. For estimation apparatus 100 according to Embodiment 2, only the configurations and processing different from those of estimation apparatus 100 according to Embodiment 1 will be specifically described, and the configurations and processing common to estimation apparatus 100 according to Embodiment 1 will not be repeatedly described in principle.

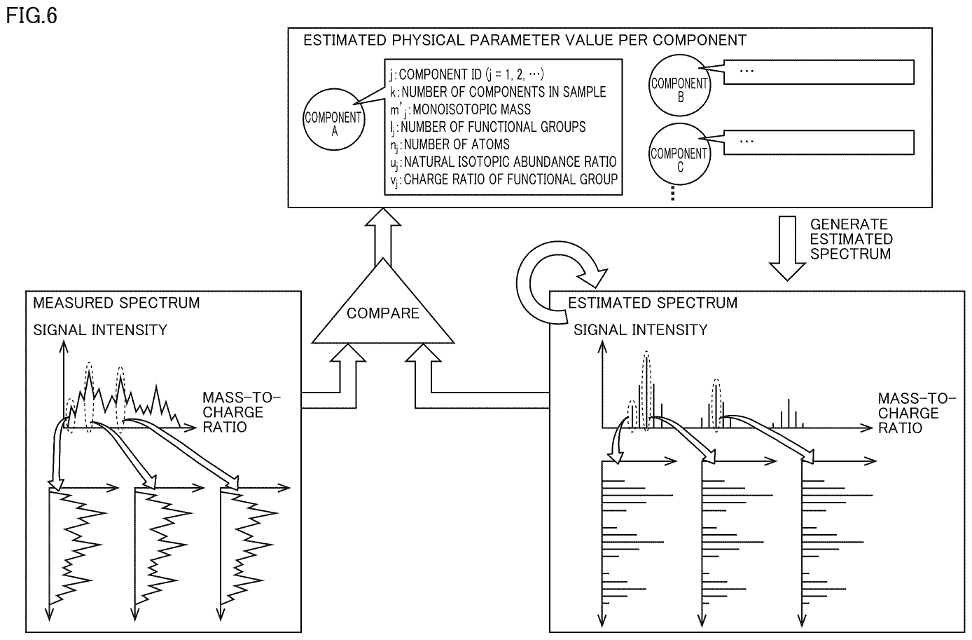

FIG. 6 is a diagram illustrating an overview of estimation processing performed by estimation apparatus 100 according to Embodiment 2. Estimation apparatus 100 according to Embodiment 1 uses a mass spectrum showing a signal intensity for a mass or a mass-to-charge ratio of an ionized component as a measured spectrum and an estimated spectrum, but estimation apparatus 100 according to Embodiment 2 uses a mass-mass spectrum as a measured spectrum and an estimated spectrum. The mass-mass spectrum is a spectrum showing a signal intensity for a mass or a mass-to-charge ratio of a fragment ion produced by decomposition of a specific ion having a specific mass or a specific mass-to-charge ratio.

For example, analysis apparatus 10 produces fragment ions by causing an inert gas to collide with a plurality of separated ionized components and separates the fragment ions according to the mass-to-charge ratio. Analysis apparatus 10 generates pulse signals corresponding to the number of fragment ions as analytical data and outputs the analytical data to estimation apparatus 100. Based on the analytical data, estimation apparatus 100 generates, as a measured spectrum, a mass-mass spectrum showing a signal intensity for a mass-to-charge ratio of the fragment ion produced by decomposition of the specific ion.

Further, estimation apparatus 100 generates an estimated spectrum of the mass-mass spectrum using physical model 135 expressed by Equation (17) below, based on a to-be-compared estimated physical parameter value.

$$[ Math 17 ]$$

$${\hat{S}}_{{msms}_{d}} ( φ ) = {\sum }_{j = 1}^{k} {\sum }_{f = 1}^{{f}_{max}} {l}_{{d}_{j} \to f} \cdot ( {U}_{{d}_{j} \to f} * R ) ( φ ) \qquad ( 17 )$$

Herein, while Equation (10) for generating an estimated mass spectrum of a mass spectrum imposes no constraints on the ion valence, the number of neutrons, and the amount of ions, Equation (17) for generating an estimated spectrum of a mass-mass spectrum imposes constraints on the valence, the number of neutrons, and the amount of ions of the fragment ions. Specifically, the valence, the number of neutrons, and the amount of ions of a fragment ion in the mass-mass spectrum are constrained to fall within the ranges of the valence, the number of neutrons, and the amount of ions of the specific ion in the mass spectrum, respectively, which serves as the basis for the fragment ion.

Estimation apparatus 100 compares the estimated spectrum of the mass-mass spectrum generated as described above with the measured spectrum of the mass-mass spectrum to calculate a posterior probability. At this time, estimation apparatus 100 performs convolution processing using a point spread function on at least one spectrum of the measured spectrum and the estimated spectrum, and then, compares these spectra to calculate a posterior probability.

As described above, estimation apparatus 100 according to Embodiment 2 generates an estimated spectrum of the mass-mass spectrum within the ranges of the valence, the number of neutrons, and the amount of ions of a specific ion in the mass spectrum, and thus, can generate an estimated spectrum of the mass-mass spectrum within the range of the characteristics of the mass spectrum. Consequently, estimation apparatus 100 can accurately estimate a physical parameter value of a component contained in a to-be-analyzed sample using the mass-mass spectrum.

### Embodiment 3

Estimation apparatus 100 according to Embodiment 3 will be described in detail with reference to the drawings. For estimation apparatus 100 according to Embodiment 3, only the configurations and processing different from those of estimation apparatuses 100 according to Embodiments 1 and 2 will be specifically described, and the configurations and processing common to estimation apparatuses 100 according to Embodiments 1 and 2 will not be repeatedly described in principle.

Estimation apparatus 100 according to Embodiment 3 is configured to estimate a physical parameter value of a component contained in a to-be-analyzed sample based on a measured spectrum, using an estimation model 140 trained by machine learning or the like.

### [Overview of Training Processing for Estimation Model]

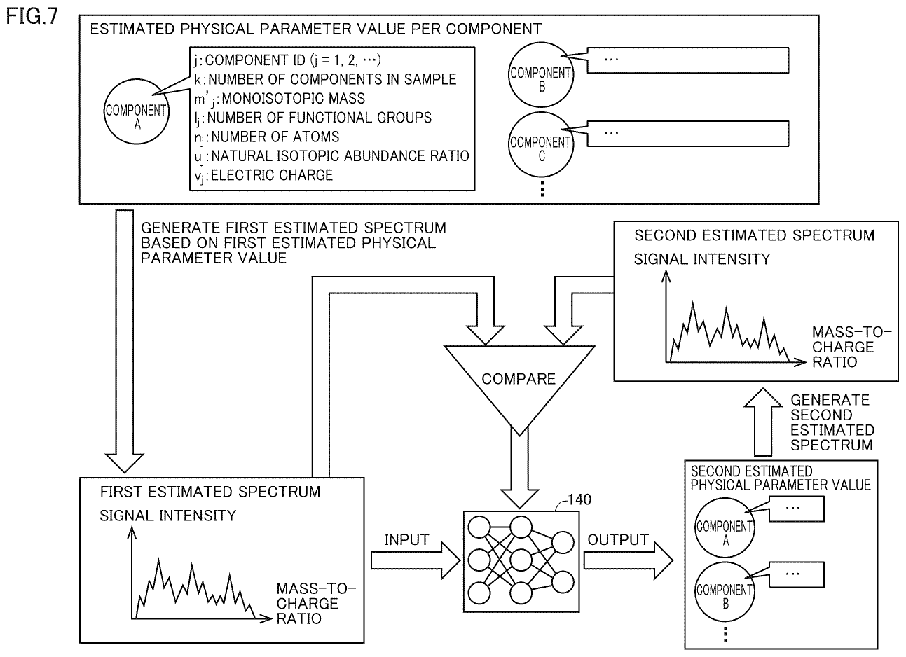

FIG. 7 is a diagram illustrating an overview of training processing for estimation model 140 performed by estimation apparatus 100 according to Embodiment 3.

As shown in FIG. 7, estimation apparatus 100 includes an estimation model 140. Estimation model 140 is stored in storage device 103. Estimation model 140 includes a neural network (not shown) and internal parameters used by the neural network.

Any algorithm that can be applied to the neural network of estimation model 140 may be applied to the neural network, such as an autoencoder, a convolutional neural network (CNN), a recurrent neural network (RNN), a transformer, or a generative adversarial network (GAN).

Estimation apparatus 100 determines the number of components and the first estimated physical parameter value (first physical parameter set). Estimation apparatus 100 generates a first estimated spectrum of the mass spectrum using physical model 135 expressed by Equation (10), based on the determined first estimated physical parameter value. Estimation apparatus 100 may generate the first estimated spectrum of the mass-mass spectrum using Equation (17).

Estimation apparatus 100 inputs the first estimated spectrum to estimation model 140 and uses estimation model 140 to estimate a second estimated physical parameter value. When the estimation accuracy of estimation model 140 is high, the second estimated physical parameter value should be closer to the first estimated physical parameter value used for generation of the first estimated spectrum.

Estimation apparatus 100 generates a second estimated spectrum of the mass spectrum using physical model 135 expressed by Equation (10), based on the second estimated physical parameter value estimated by estimation model 140. When having generated the first estimated spectrum using Equation (17), estimation apparatus 100 may generate the second estimated spectrum of the mass-mass spectrum using Equation (17).

Further, when generating the first estimated spectrum and the second estimated spectrum of the mass-mass spectrum, estimation apparatus 100 may impose constraints on the valence, the number of neutrons, and the amount of ions of the fragment ion in the mass-mass spectrum to fall within the ranges of the valence, the number of neutrons, and the amount of ions of the specific ion in the mass spectrum, respectively, which serves as the basis for the fragment ion.

Estimation apparatus 100 compares the first estimated spectrum with the second estimated spectrum to calculate a posterior probability. At this time, estimation apparatus 100 performs convolution processing using a point spread function that increases the width of at least one spectrum of the first estimated spectrum and the second estimated spectrum, and then, compares the spectra to calculate a posterior probability. For example, estimation apparatus 100 may transform the shape of at least one spectrum of the first estimated spectrum and the second estimated spectrum into a Gaussian shape by performing convolution processing using a point spread function on the at least one spectrum. Estimation apparatus 100 may perform convolution processing using a point spread function only on the first estimated spectrum, perform convolution processing using a point spread function only on the second estimated spectrum, or perform convolution processing using a point spread function on each of the first estimated spectrum and the second estimated spectrum.

Estimation apparatus 100 trains estimation model 140 based on the comparison result (posterior probability) between the first estimated spectrum and the second estimated spectrum. Specifically, estimation apparatus 100 inputs the calculated posterior probability to estimation model 140. Estimation model 140 updates (machine learning) internal parameters (not shown) based on the feedback-input posterior probability.

Estimation apparatus 100 repeats the processing of FIG. 7 described above while varying the to-be-compared number of components and the first estimated physical parameter value to train estimation model 140 such that estimation model 140 can accurately estimate the second estimated physical parameter value close to the first estimated physical parameter value.

### [Flow of Training Processing of Estimation Apparatus]

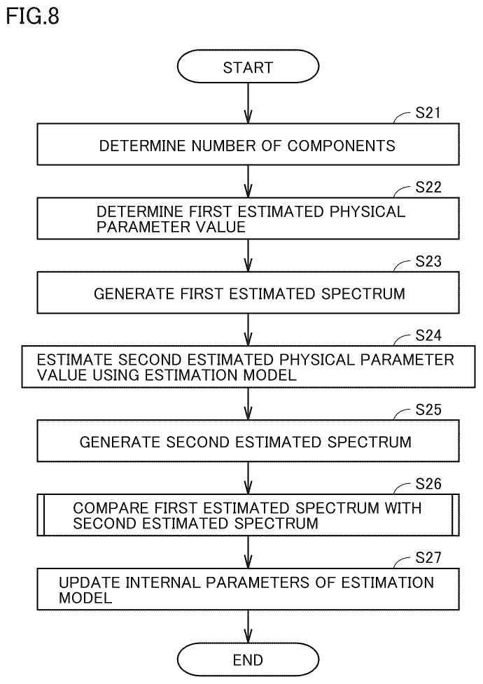

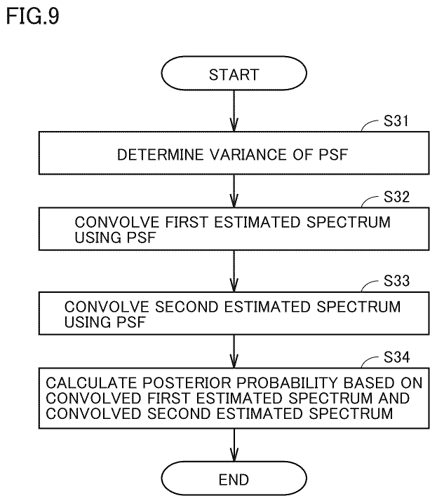

FIG. 8 is a flowchart of a main process in training processing performed by estimation apparatus 100 according to Embodiment 3. FIG. 9 is a flowchart of a subprocess in the training processing performed by estimation apparatus 100 according to Embodiment 3. Each processing step (hereinafter abbreviated as “S”) shown in FIGS. 8 and 9 is realized by computing device 101 executing estimation processing program 130. Estimation apparatus 100 performs the processing shown in FIGS. 8 and 9 during the training of estimation model 140.

As shown in FIG. 8, estimation apparatus 100 determines the number of components (S21). For example, for the first estimation, estimation apparatus 100 determines “1” as the number of components. Estimation apparatus 100 determines a first estimated physical parameter value for the number of components (S22).

Estimation apparatus 100 generates a first estimated spectrum based on the determined first estimated physical parameter value (S23). For example, estimation apparatus 100 generates a first estimated spectrum showing a signal intensity corresponding to the mass-to-charge ratio based on the mass and charge, which are the first estimated physical parameter values.

Estimation apparatus 100 inputs the first estimated spectrum to estimation model 140 and estimates a second estimated physical parameter value using estimation model 140 (S24). Estimation apparatus 100 generates a second estimated spectrum based on the second estimated physical parameter value estimated by estimation model 140 (S25). Estimation apparatus 100 compares the first estimated spectrum generated in S23 with the second estimated spectrum generated in S25 (S26).

The subprocess of the processing in S26 is shown in FIG. 9. Specifically, as shown in FIG. 9, estimation apparatus 100 determines a variance of the point spread function (PSF) (S31).

Estimation apparatus 100 performs convolution processing using a point spread function on at least one spectrum of the first estimated spectrum and the second estimated spectrum. Estimation apparatus 100 performs convolution processing using a point spread function on the first estimated spectrum to transform the first estimated spectrum into a Gaussian shape, thereby increasing the width of the first estimated spectrum in the horizontal axis direction (S32). Further, estimation apparatus 100 performs convolution processing using a point spread function on the second estimated spectrum to transform the second estimated spectrum into a Gaussian shape, thereby increasing the width of the second estimated spectrum in the horizontal axis direction (S33).

After the convolution processing, estimation apparatus 100 compares the first estimated spectrum with the second estimated spectrum to calculate a posterior probability (S34). For example, estimation apparatus 100 calculates a posterior probability based on the first estimated spectrum after the convolution processing and the second estimated spectrum after the convolution processing.

Returning to FIG. 8, estimation apparatus 100 trains estimation model 140 by updating the internal parameters of estimation model 140 based on the comparison result between the first estimated spectrum and the second estimated spectrum (S27). Subsequently, estimation apparatus 100 terminates the training processing shown in FIG. 8.

### [Overview of Estimation Processing for Estimation Model]

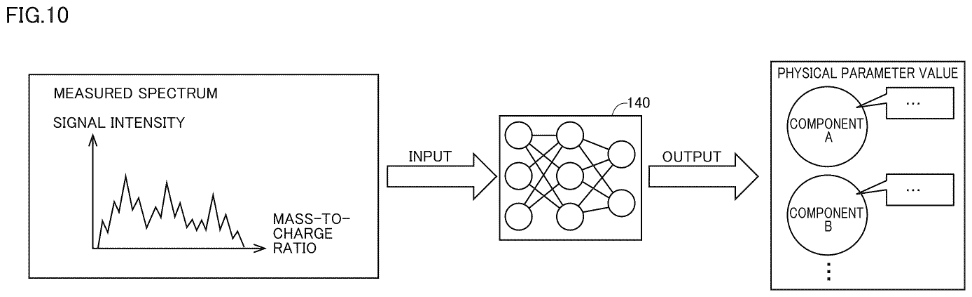

FIG. 10 is a diagram illustrating an overview of estimation processing of estimation model 140 performed by estimation apparatus 100 according to Embodiment 3.

As shown in FIG. 10, an analyst actually measures a to-be-analyzed sample using analysis apparatus 10. Analytical data from analysis apparatus 10 is input to estimation apparatus 100. Based on the analytical data acquired from analysis apparatus 10, estimation apparatus 100 generates a measured spectrum indicating a signal intensity for a spectral parameter value. In the example of FIG. 10, estimation apparatus 100 generates a mass spectrum as the measured spectrum.

Estimation apparatus 100 inputs the measured spectrum to estimation model 140 and uses estimation model 140 to estimate a physical parameter value of a component contained in the to-be-analyzed sample. If the estimation accuracy of estimation model 140 has become higher through training, the estimated physical parameter value estimated by estimation model 140 should be closer to the physical parameter value of the component contained in the to-be-analyzed sample.

### [Flow of Estimation Processing of Estimation Apparatus]

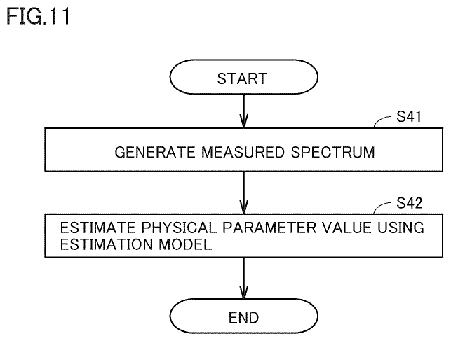

FIG. 11 is a flowchart of estimation processing performed by estimation apparatus 100 according to Embodiment 3. Each processing step (hereinafter abbreviated as “S”) shown in FIG. 11 is realized by computing device 101 executing estimation process program 130. Estimation apparatus 100 performs the processing shown in FIG. 11 when estimating a physical parameter value of a component contained in a sample using estimation model 140.

As shown in FIG. 11, estimation apparatus 100 generates a measured spectrum indicating a signal intensity for a spectral parameter value based on analytical data acquired from analysis apparatus 10 (S41). Estimation apparatus 100 inputs the measured spectrum to estimation model 140 and uses estimation model 140 to estimate a physical parameter value of a component contained in a to-be-analyzed sample (S42).

As described above, estimation apparatus 100 according to Embodiment 3 can estimate the physical parameter value of the component of the to-be-analyzed sample using estimation model 140 based on the measured spectrum obtained by measuring the sample. Further, during training of estimation model 140, when comparing the first estimation spectrum with the second estimation spectrum, estimation apparatus 100 performs convolution processing using a point spread function on at least one spectrum of the first estimated spectrum and the second estimated spectrum, and then, compares the spectra with each other. This enables estimation apparatus 100 to avoid a situation in which the gradient of the likelihood function vanishes, making it impossible to search for an optimal physical parameter value. Consequently, estimation apparatus 100 can train estimation model 140 without increasing a computational cost, accurately estimating the physical parameter value of the component contained in the to-be-analyzed sample.

### Aspects

It will be appreciated by a person skilled in the art that the exemplary embodiments described above provide specific examples of the following aspects.
(Clause 1) An estimation apparatus according to an aspect includes: a data acquisition unit that acquires analytical data of a component; a computing unit that estimates a physical parameter value of the component based on the analytical data acquired by the data acquisition unit; and a display unit that displays the physical parameter value of the component estimated by the computing unit. The computing unit: generates a measured spectrum indicating a signal intensity for a spectrum parameter value based on the analytical data; generates an estimated spectrum indicating a signal intensity for the spectrum parameter value based on an estimated physical parameter value; performs convolutional processing using a point spread function, which increases a width of at least one spectrum of the measured spectrum and the estimated spectrum to a first width, and compares the measured spectrum with the estimated spectrum; and estimates the physical parameter value of the component based on a comparison result between the measured spectrum and the estimated spectrum.

The estimation apparatus according to clause 1 can compare the estimated spectrum estimated using a model based on the estimated physical parameter value with the measured spectrum obtained by measuring a to-be-analyzed sample to estimate the number of components and a physical parameter value of the to-be-analyzed sample. Further, when comparing the estimated spectrum with the measured spectrum, the estimation apparatus performs convolutional processing using a point spread function on at least one spectrum of the measured spectrum and the estimated spectrum, and then, compares the spectra with each other. This enables the estimation apparatus to avoid a situation in which the gradient of the likelihood function vanishes, making is impossible to search for an optimal physical parameter value. Consequently, the estimation apparatus can accurately estimate a physical parameter value of a component contained in a to-be-analyzed sample, without increasing a computational cost.
(Clause 2) In the estimation apparatus according to clause 1, the convolutional processing includes processing of transforming a shape of the at least one spectrum into a Gaussian shape. The computing unit performs the convolutional processing and overlays at least part of the measured spectrum on the estimated spectrum.

The estimation apparatus according to clause 2 can perform convolutional processing and overlays at least part of the measured spectrum on the estimated spectrum, thereby comparing the measured spectrum with the estimated spectrum.
(Clause 3) In the estimation apparatus according to clause 2, the computing unit transforms the shape of the at least one spectrum by the convolutional processing into the Gaussian shape to increase the width of the at least one spectrum.

The estimation apparatus according to clause 3 can perform convolutional processing to transform a spectrum into a Gaussian shape, thereby increasing the width of the spectrum. Thus, at least part of the measured spectrum can be overlaid on the estimated spectrum.
(Clause 4) The estimation apparatus according to any one of clauses 1 to 3 further includes a storage unit that stores a model function that generates the estimated spectrum based on the estimated physical parameter value. The computing unit generates the estimated spectrum based on the estimated physical parameter value using the model function.

The estimation apparatus according to clause 4 can generate an estimated spectrum based on the estimated physical parameter value using the model function.
(Clause 5) In the estimation apparatus according to any one of clauses 1 to 4, the computing unit determines, based on the comparison result, another estimated physical parameter value from a plurality of physical parameter values determined by a prior distribution, and generates another estimated spectrum based on the other estimated physical parameter value using a model function.

The estimation apparatus according to clause 5 can determine, based on a comparison result between the measured spectrum and the other estimated spectrum, an optimal estimated physical parameter value used for generation of the other estimated spectrum. Thus, a physical parameter value of a component contained in a to-be-analyzed sample can be estimated accurately.
(Clause 6) In the estimation apparatus according to any one of clauses 1 to 5, the computing unit performs convolutional processing using a point spread function, which increases a width of at least one spectrum of the measured spectrum and the other estimated spectrum to a second width smaller than the first width, and compares the measured spectrum with the other estimated spectrum, and estimates the physical parameter value of the component based on a comparison result between the measured spectrum and the other estimated spectrum.

The estimation apparatus according to clause 6 can search for an optimal estimated physical parameter value for generating an estimated spectrum that is closest to the measured spectrum while gradually narrowing the Gaussian shape.
(Clause 7) In the estimation apparatus according to any one of clauses 1 to 6, the estimated physical parameter value includes at least one value of a monoisotopic mass, an electric charge, a number of functional groups, a charging rate of a functional group, a number of atoms, and a natural isotopic abundance ratio.

The estimation apparatus according to clause 7 can estimate at least one physical parameter value of a monoisotopic mass, an electric charge, a number of functional groups, a charging rate of a functional group, a number of atoms, and a natural isotopic abundance ratio.
(Clause 8) In the estimation apparatus according to any one of clauses 1 to 7, the spectrum parameter value is a mass or a mass-to-charge ratio of the component ionized. Each of the measured spectrum and the estimated spectrum is a mass spectrum.

The estimation apparatus according to clause 8 can generate an estimated spectrum of a mass spectrum to estimate a physical parameter value of a component contained in a to-be-analyzed sample.
(Clause 9) In the estimation apparatus according to any one of clauses 1 to 7, the spectrum parameter value is a mass or a mass-to-charge ratio of a fragment ion produced by decomposition of a specific ion having a specific mass or a specific mass-to-charge ratio. Each of the measured spectrum and the estimated spectrum is a mass-mass spectrum.

The estimation apparatus according to clause 9 can generate an estimated spectrum of a mass-mass spectrum to estimate a physical parameter value of a component contained in a to-be-analyzed sample.
(Clause 10) In the estimation apparatus according to clause 9, the computing unit generates the mass-mass spectrum using a mass spectrum indicating a signal intensity for a mass or a mass-to-charge ratio of the component ionized. A valence, a number of neutrons, and an amount of ions of the fragment ion in the mass-mass spectrum are within ranges of a valence, a number of neutrons, and an amount of ions of the specific ion in the mass spectrum, respectively.

The estimation apparatus according to clause 10 generates an estimated spectrum of a mass-mass spectrum within the ranges of the valence, the number of neutrons, and the amount of ions of the specific ion in the mass spectrum, and thus, can generate an estimated spectrum of the mass-mass spectrum within the range of characteristics of the mass spectrum. This enables the estimation apparatus to accurately estimate a physical parameter value of a component contained in a to-be-analyzed sample using the mass-mass spectrum.
(Clause 11) An estimation method according to an aspect includes: generating a measured spectrum indicating a signal intensity for a spectrum parameter value based on analytical data of a component; generating an estimated spectrum indicating a signal intensity for the spectrum parameter value based on an estimated physical parameter value; performing convolutional processing using a point spread function, which increases a width of at least one spectrum of the measured spectrum and the estimated spectrum to a first width, and comparing the measured spectrum with the estimated spectrum; and estimating the physical parameter value of the component based on a comparison result between the measured spectrum and the estimated spectrum.

The estimation method according to clause 11 can compare an estimated spectrum estimated using a model based on the estimated physical parameter value with a measured spectrum obtained by measuring a to-be-analyzed sample to estimate the number of components and a physical parameter value of the to-be-analyzed sample. Further, when comparing the estimated spectrum with the measured spectrum, the estimation apparatus performs convolutional processing using a point spread function on at least one spectrum of the measured spectrum and the estimated spectrum, and then, compares the spectra with each other. This enables the estimation apparatus to avoid a situation in which the gradient of the likelihood function vanishes, making it impossible to search for an optimal physical parameter value. Consequently, the estimation apparatus can accurately estimate a physical parameter value of a component contained in a to-be-analyzed sample, without increasing a computational cost.
(Clause 12) An estimation apparatus according to an aspect includes: a data acquisition unit that acquires analytical data of a component; a computing unit that estimates a physical parameter value of the component based the analytical data acquired by the data acquisition unit; and a display unit that displays the physical parameter value of the component estimated by the computing unit. In estimation of the physical parameter value of the component, the computing unit generates a measured spectrum indicating a signal intensity for a spectrum parameter value based on the analytical data, and estimates the physical parameter value of the component based on the measured spectrum using an estimation model. In training of the estimation model, the computing unit: determines a first estimated physical parameter value; generates, based on the first estimated physical parameter value, a first estimated spectrum indicating a signal intensity for the spectrum parameter value; estimates a second estimated physical parameter value based on the first estimated spectrum using the estimation model; generates, based on the second estimated physical parameter value, a second estimated spectrum indicating a signal intensity for the spectrum parameter value; performs convolutional processing using a point spread function, which increases a width of at least one spectrum of the first estimated spectrum and the second estimated spectrum, and compares the first estimated spectrum with the second estimated spectrum; and trains the estimation model based on a comparison result between the first estimated spectrum and the second estimated spectrum.

The estimation apparatus according to clause 12 can estimate a physical parameter value of a to-be-analyzed sample using an estimation model based on a measured spectrum obtained by measuring the sample. Further, in training of the estimation model, when comparing the first estimated spectrum with the second estimated spectrum, the estimation apparatus performs convolutional processing using a point spread function on at least one spectrum of the first estimated spectrum and the second estimated spectrum, and then, compares the spectra with each other. This enables the estimation apparatus to avoid a situation in which the gradient of the likelihood function vanishes, making it impossible to search for an optimal physical parameter value. Consequently, the estimation apparatus can train the estimation model without increasing a computational cost, accurately estimating a physical parameter value of a component contained in a to-be-analyzed sample using the estimation model.
(Clause 13) In the estimation apparatus according to clause 12, the convolutional processing includes processing of transforming a shape of the at least one spectrum into a Gaussian shape. The computing unit performs the convolutional processing and overlays at least part of the first estimated spectrum on the second estimated spectrum.

The estimation apparatus according to clause 13 can perform convolutional processing and overlays at least part of the first estimated spectrum on the second estimated spectrum, thereby comparing the first estimated spectrum with the second estimated spectrum.
(Clause 14) In the estimation apparatus according to clause 13, the computing unit transforms the shape of the at least one spectrum into the Gaussian shape by the convolutional processing to increase the width of the at least one spectrum.

The estimation apparatus according to clause 14 can perform convolutional processing to increase the width of the Gaussian shape of the spectrum, overlaying at least part of the measured spectrum on the estimated spectrum.
(Clause 15) In the estimation apparatus according to any one of clauses 12 to 14, each of the first estimated physical parameter value and the second estimated physical parameter value includes at least one value of a monoisotopic mass, an electric charge, a number of functional groups, a charging rate of a functional group, a number of atoms, and a natural isotopic abundance ratio.

The estimation apparatus according to clause 15 can estimate at least one physical parameter value of a monoisotopic mass, an electric charge, a number of functional groups, a charging rate of a functional group, a number of atoms, and a natural isotopic abundance ratio.
(Clause 16) In the estimation apparatus according to any one of clauses 12 to 15, the spectrum parameter value is a mass or a mass-to-charge ratio of the component ionized. Each of the measured spectrum, the first estimated spectrum, and the second estimated spectrum is a mass spectrum.

The estimation apparatus according to clause 16 can train an estimation model using the first estimated spectrum and the second estimated spectrum of the mass spectrum.
(Clause 17) In the estimation apparatus according to any one of clauses 12 to 15, the spectrum parameter value is a mass or a mass-to-charge ratio of a fragment ion produced by decomposition of a specific ion having a specific mass or a specific mass-to-charge ratio. Each of the first estimated spectrum and the second estimated spectrum is a mass-mass spectrum.

The estimation apparatus according to clause 17 can train an estimation model using the first estimated spectrum and the second estimated spectrum of the mass-mass spectrum.
(Clause 18) In the estimation apparatus according to clause 17, the computing unit generates the mass-mass spectrum using a mass spectrum indicating a signal intensity for a mass or a mass-to-charge ratio of the component ionized. A valence, a number of neutrons, and an amount of ions of the fragment ion in the mass-mass spectrum are within ranges of a valence, a number of neutrons, and an amount of ions of the specific ion in the mass spectrum, respectively

The estimation apparatus according to clause 18 generates an estimated spectrum of a mass-mass spectrum within the ranges of the valence, the number of neutrons, and the amount of ions of the specific ion in the mass spectrum, and thus, can generate an estimated spectrum of a mass-mass spectrum within the range of characteristics of the mass spectrum. This enables the estimation apparatus to accurately estimate a physical parameter value of a component contained in a to-be-analyzed sample using a mass-mass spectrum.
(Clause 19) An estimation method according to an aspect includes, in estimation of a physical parameter value of a component: generating, based on analytical data of the component, a measured spectrum indicating a signal intensity for a spectrum parameter value; and estimating the physical parameter value of the component based on the measured spectrum using an estimation model. The estimation method includes, in training of the estimation model: determining a first estimated physical parameter value; generating, based on the first estimated physical parameter value, a first estimated spectrum indicating a signal intensity for the spectrum parameter value; estimating a second estimated physical parameter value based on the first estimated spectrum using the estimation model; generating, based on the second estimated physical parameter value, a second estimated spectrum indicating a signal intensity for the spectrum parameter value; performing convolutional processing using a point spread function, which increases a width of at least one spectrum of the first estimated spectrum and the second estimated spectrum, and comparing the first estimated spectrum with the second estimated spectrum; and training the estimation model based on a comparison result between the first estimated spectrum and the second estimated spectrum.

The estimation method according to clause 19 can estimate, based on the measured spectrum obtained by measuring a to-be-analyzed sample, a physical parameter value of a component of the sample using the estimation model. Further, in training of the estimation model, when comparing the first estimation spectrum with the second estimation spectrum, the estimation method performs convolution processing using a point spread function on at least one spectrum of the first estimation spectrum and the second estimation spectrum, and then, compares the spectra with each other. The estimation method can thus avoid a situation in which the gradient of the likelihood function vanishes, making it impossible to search for an optimal physical parameter value. Consequently, the estimation method can train the estimation model without increasing a computational cost, accurately estimating a physical parameter value of a component contained in a to-be-analyzed sample.

Although the embodiments of the present invention have been described, it should be understood that the embodiments disclosed herein are illustrative and non-restrictive in every respect. The scope of the present invention is defined by the terms of the claims and is intended to include any modifications within the scope and meaning equivalent to the terms of the claims.

What is claimed is:

1. An estimation apparatus that estimates a physical parameter value of a component contained in a sample, the estimation apparatus comprising:
a data acquisition unit that acquires analytical data of the component;
a computing unit that estimates the physical parameter value of the component based on the analytical data acquired by the data acquisition unit; and
a display unit that displays the physical parameter value of the component estimated by the computing unit,
wherein the computing unit
generates a measured spectrum indicating a signal intensity for a spectrum parameter value based on the analytical data,
generates an estimated spectrum indicating a signal intensity for the spectrum parameter value based on an estimated physical parameter value,
performs convolutional processing using a point spread function and compares the measured spectrum with the estimated spectrum, the point spread function increasing a width of at least one spectrum of the measured spectrum and the estimated spectrum to a first width, and
estimates the physical parameter value of the component based on a comparison result between the measured spectrum and the estimated spectrum.

2. The estimation apparatus according to claim 1, wherein
the convolutional processing includes processing of transforming a shape of the at least one spectrum into a Gaussian shape, and
the computing unit performs the convolutional processing and overlays at least part of the measured spectrum on the estimated spectrum.

3. The estimation apparatus according to claim 2, wherein the computing unit transforms the shape of the at least one spectrum by the convolutional processing into the Gaussian shape to increase the width of the at least one spectrum.

4. The estimation apparatus according to claim 1, further comprising a storage unit that stores a model function that generates the estimated spectrum based on the estimated physical parameter value,
wherein the computing unit generates the estimated spectrum based on the estimated physical parameter value using the model function.

5. The estimation apparatus according to claim 4, wherein the computing unit
determines, based on the comparison result, another estimated physical parameter value from a plurality of physical parameter values determined by a prior distribution, and
generates another estimated spectrum based on the other estimated physical parameter value using the model function.

6. The estimation apparatus according to claim 5, wherein the computing unit
performs convolutional processing using a point spread function and compares the measured spectrum with the other estimated spectrum, the point spread function increasing a width of at least one spectrum of the measured spectrum and the other estimated spectrum to a second width smaller than the first width, and
estimates the physical parameter value of the component based on a comparison result between the measured spectrum and the other estimated spectrum.

7. The estimation apparatus according to claim 1, wherein the estimated physical parameter value includes at least one value of a monoisotopic mass, an electric charge, a number of functional groups, a charging rate of a functional group, a number of atoms, and a natural isotopic abundance ratio.

8. The estimation apparatus according to claim 1, wherein
the spectrum parameter value is a mass or a mass-to-charge ratio of the component ionized, and
each of the measured spectrum and the estimated spectrum is a mass spectrum.

9. The estimation apparatus according to claim 1, wherein
the spectrum parameter value is a mass or a mass-to-charge ratio of a fragment ion produced by decomposition of a specific ion having a specific mass or a specific mass-to-charge ratio, and
each of the measured spectrum and the estimated spectrum is a mass-mass spectrum.

10. The estimation apparatus according to claim 9, wherein
the computing unit generates the mass-mass spectrum using a mass spectrum indicating a signal intensity for a mass or a mass-to-charge ratio of the component ionized, and
a valence, a number of neutrons, and an amount of ions of the fragment ion in the mass-mass spectrum are within ranges of a valence, a number of neutrons, and an amount of ions of the specific ion in the mass spectrum, respectively.

11. An estimation method for estimating, by a computer, a physical parameter value of a component contained in a sample, the estimation method comprising:
generating a measured spectrum indicating a signal intensity for a spectrum parameter value based on analytical data of the component;
generating an estimated spectrum indicating a signal intensity for the spectrum parameter value based on an estimated physical parameter value;
performing convolutional processing using a point spread function and comparing the measured spectrum with the estimated spectrum, the point spread function increasing a width of at least one spectrum of the measured spectrum and the estimated spectrum to a first width; and
estimating the physical parameter value of the component based on a comparison result between the measured spectrum and the estimated spectrum.

12. An estimation apparatus that estimates a physical parameter value of a component contained in a sample, the estimation apparatus comprising:
a data acquisition unit that acquires analytical data of the component;
a computing unit that estimates the physical parameter value of the component based on the analytical data acquired by the data acquisition unit; and
a display unit that displays the physical parameter value of the component estimated by the computing unit, wherein
in estimation of the physical parameter value of the component, the computing unit
generates a measured spectrum indicating a signal intensity for a spectrum parameter value based on the analytical data, and
estimates the physical parameter value of the component based on the measured spectrum using an estimation model, and
in training of the estimation model, the computing unit
determines a first estimated physical parameter value,
generates, based on the first estimated physical parameter value, a first estimated spectrum indicating a signal intensity for the spectrum parameter value,
estimates a second estimated physical parameter value based on the first estimated spectrum using the estimation model,
generates, based on the second estimated physical parameter value, a second estimated spectrum indicating a signal intensity for the spectrum parameter value,
performs convolutional processing using a point spread function and compares the first estimated spectrum with the second estimated spectrum, the point spread function increasing a width of at least one spectrum of the first estimated spectrum and the second estimated spectrum, and
trains the estimation model based on a comparison result between the first estimated spectrum and the second estimated spectrum.

13. The estimation apparatus according to claim 12, wherein
the convolutional processing includes processing of transforming a shape of the at least one spectrum into a Gaussian shape, and
the computing unit performs the convolutional processing and overlays at least part of the first estimated spectrum on the second estimated spectrum.

14. The estimation apparatus according to claim 13, wherein the computing unit transforms the shape of the at least one spectrum into the Gaussian shape by the convolutional processing to increase the width of the at least one spectrum.

15. The estimation apparatus according to claim 12, wherein each of the first estimated physical parameter value and the second estimated physical parameter value includes at least one value of a monoisotopic mass, an electric charge, a number of functional groups, a charging rate of a functional group, a number of atoms, and a natural isotopic abundance ratio.

16. The estimation apparatus according to claim 12, wherein
the spectrum parameter value is a mass or a mass-to-charge ratio of the component ionized, and
each of the measured spectrum, the first estimated spectrum, and the second estimated spectrum is a mass spectrum.

17. The estimation apparatus according to claim 12, wherein
the spectrum parameter value is a mass or a mass-to-charge ratio of a fragment ion produced by decomposition of a specific ion having a specific mass or a specific mass-to-charge ratio, and
each of the first estimated spectrum and the second estimated spectrum is a mass-mass spectrum.

18. The estimation apparatus according to claim 17, wherein
the computing unit generates the mass-mass spectrum using a mass spectrum indicating a signal intensity for a mass or a mass-to-charge ratio of the component ionized, and
a valence, a number of neutrons, and an amount of ions of the fragment ion in the mass-mass spectrum are within ranges of a valence, a number of neutrons, and an amount of ions of the specific ion in the mass spectrum, respectively.
